(19) United States
(12) Patent Application Publication
(10) Pub. No.: US 2012/0251638 A1
(43) Pub. Date: Oct. 4, 2012

(54) Nutritional Supplement Comprising Nut Oil Composition

(76) Inventor: Andrew Baker, Danvers, MA (US)

(21) Appl. No.: 13/075,118

(22) Filed: Mar. 29, 2011

(51) Int. Cl. A61K 36/736; A61P 27/02; A61K 31/355; A61P 3/06; A61K 31/20; A61K 31/201

(52) U.S. Cl. 424/735; 514/558; 514/560; 514/458

(57) ABSTRACT

The present invention provides a novel composition of nut oils, specifically; walnut oil, almond oil, avocado oil and pistachio oil for use in maintaining and improving health in a convenient and cost-effective manner; for obtaining essential fatty acids that are required for normal body functioning; for reducing cholesterol, and for reducing retinal degeneration in an individual with retinitis pigmentosa.

### BACKGROUND OF THE INVENTION

Diet is often an important factor in maintaining and improving health. In general, a healthy diet is important for the prevention of many chronic health risks such as obesity, heart disease, diabetes, and cancer. A healthy diet often includes proper amounts of essential nutrients which are nutrients required for normal body functioning that either cannot be synthesized by the body at all or cannot be synthesized in amounts adequate for good health and thus is obtained from a nutritional or dietary source. Essential nutrients include, for example, vitamins, dietary minerals, essential amino acids and essential fatty acids.

While a number of dietary and nutritional supplements are available, many do not contain effective amounts and/or ingredients and often contain side effects.

Hence, a need exists for an effective nutritional supplement containing a novel combination of nut oils for maintaining and improving health and for obtaining essential fatty acids that are needed for normal body functioning. Furthermore, a need exists for doing so in a convenient and cost effective manner.

### SUMMARY OF THE INVENTION

The present invention provides a novel nut oil composition for use in maintaining and improving health. The present invention further provides a novel nut oil composition for obtaining essential fatty acids that are required for normal body functioning. The nut oil composition of the present invention provides a novel combination of walnut oil, almond oil, avocado oil and pistachio oil to maintain and improve health and to obtain essential fatty acids that are not produced by the body or which a limited amount are produced by the body. The nut oil composition contains walnut oil in an amount ranging between about 10% to about 35% by weight in said composition, almond oil in an amount ranging between about 10% to about 35% by weight in said composition, avocado oil in an amount ranging between about 10% to about 35% by weight in said composition and pistachio oil in an amount ranging between about 10% to about 35% by weight in said composition. In an embodiment, the carrier comprises a softgel capsule, a capsule, a caplet, a pill, liquid formulation or any combination thereof. In yet another embodiment, the ratio of walnut oil, the ratio of almond oil, the ratio of avocado oil and the ratio of pistachio oil in the composition is in the range of 1:1:1:1.

Additionally, the novel combination of walnut oil, almond oil, avocado oil and pistachio oil in the present invention when combined together contains the following fatty acids selected from the group consisting of palmitic acid, palmitoleic acid, heptadecanoic acid, stearic acid, arachidic acid, eicosenoic acid, linoleic acid, linolenic acid, oleic acid or any combination thereof. Furthermore, the nutritional supplement of the present invention can include vitamin E, omega-3, omega-6, omega-7, omega-9, an antioxidant or any combination thereof.

The composition of the present invention includes a nut oil composition having walnut oil in an amount ranging between about 100 mg/mL and about 350 mg/mL; almond oil in an amount ranging between about 100 mg/mL and about 350 mg/mL; an avocado oil in an amount ranging between about 100 mg/mL and about 350 mg/mL; and pistachio oil in an amount ranging between about 100 mg/mL and about 350 mg/mL. The composition of the present invention can further include further vitamin E in an amount of between about 10% and about 15% by weight.

The present invention pertains to, in an aspect, a composition having an amount of omega-3 in the range of about 2.0% to about 10% of total lipids in composition; an amount of omega-6 in the range of about 25% to about 45% of total lipids in composition; an amount of omega-7 in the range of about 0.1% and about 5% of total lipids in composition; and an amount of omega-9 in the range about 40% and about 60% of total lipids in composition.

The nut oil composition of the present invention embodies palmitic acid in an amount ranging between about 5 to about 20% by weight; stearic acid in an amount ranging between about 0.5% to about 10% by weight; oleic acid in an amount ranging between about 40% and about 60% by weight; linoleic acid in an amount ranging between about 25% to about 45% by weight; alpha linolenic acid in an amount ranging between about 2% and about 10% by weight; palmitoleic acid in an amount ranging between about 0.1% and about 5% by weight; heptadecanoic acid in an amount ranging between about 0.1% and about 0.25% by weight; arachidic acid in an amount ranging between about 0.1% and about 0.25% by weight; eicosenoic acid in an amount between about 0.1% and about 0.25% by weight, and a combination thereof.

The present invention relates to methods of treating an individual with a high total cholesterol level, high blood lipid levels (e.g., LDL-C, HDL-C) or both in an individual, by selecting an individual having elevated total cholesterol levels, abnormal blood lipid levels or both; and administering to the individual an effective amount of nut oil composition comprising; a walnut oil in an amount ranging between about 10% and about 35% by weight; an almond oil in an amount ranging between about 10% and about 35% by weight; an avocado oil in an amount ranging between about 10% to about 35% by weight; and a pistachio oil in an amount ranging between about 10% to about 35% by weight to decreases the total cholesterol levels, blood lipid levels or both. In one embodiment, the present invention pertains to selecting individuals that also have total-C levels and/or LDL-C level(s) that are considered to be high or borderline high (e.g., individuals with levels on the higher side of a normal ranges). As such, the present invention embodies administering the nut oil composition to an individual having a Total-C of greater than about 200 mg/dL (e.g., between about 200 mg/dL and about 350 mg/dL) and/or an LDL-C of greater than about 130 mg/dL (e.g., between about 130 mg/dL and 280 mg/dL). Additionally, the present invention embodies administering the nut oil composition to an individual with high triglyceride levels, e.g., administering the nut oil composition to an individual with a triglyceride level of greater than 150 mg/dL (e.g., between about 150 mg/dL and about 300 mg/dL). As described herein, total cholesterol levels, blood lipid levels or both is decreased by at least 5% (e.g., 10%, 15%, 20%, 25%, 30%, 35%, 40%, 50%, 60%, 70%, 80%, or 90%) as compared to the level prior to administering the nut oil formulation described herein.

The present invention also relates to methods of reducing retinal degeneration in an individual with retinitis pigmentosa, by selecting an individual having retinitis pigmentosa and administering to the individual an effective amount of nut oil composition comprising; a walnut oil in an amount ranging between about 10% and about 35% by weight; an almond oil in an amount ranging between about 10% and about 35% by weight; an avocado oil in an amount ranging between about 10% to about 35% by weight; and a pistachio oil in an amount ranging between about 10% to about 35% by weight to reduce retinal degeneration. In an embodiment, the present invention pertains to selecting an individual with retinitis pigmentosa and administering the nut oil composition to the individual wherein the retinal degeneration is reduced by at least 5% (e.g., 10%, 15%, 20%, 25%, 30%, 35%, 40%, 50%, 60%, 70%, 80%, or 90%) as compared to the level prior to administering the nut oil formulation described herein.

Yet the present invention also relates to methods of reducing the decline in visual field sensitivity in an individual with retinitis pigmentosa, by selecting an individual having retinitis pigmentosa and administering to the individual an effective amount of nut oil composition comprising; a walnut oil in an amount ranging between about 10% and about 35% by weight; an almond oil in an amount ranging between about 10% and about 35% by weight; an avocado oil in an amount ranging between about 10% to about 35% by weight; and a pistachio oil in an amount ranging between about 10% to about 35% by weight to reduce the decline of visual sensitivity. In an embodiment, the present invention pertains to selecting an individual with retinitis pigmentosa and administering the nut oil composition to the individual wherein the decline of visual sensitivity is reduced by at least 5% (e.g., 10%, 15%, 20%, 25%, 30%, 35%, 40%, 50%, 60%, 70%, 80%, or 90%) as compared to the level prior to administering the nut oil formulation described herein.

The present invention has a number of advantages over other nutritional and dietary supplements. The present invention provides a novel composition of nut oils, specifically; walnut oil, almond oil, avocado oil and pistachio oil for use in maintaining and improving health in a convenient and cost-effective manner. The present invention allows for a wide variety of essential fatty acids in a single supplement. The supplement of the present invention advantageously allows for treating high cholesterol, reducing retinal degeneration with retinitis pigmentosa and for reducing the decline in visual field sensitivity.

### BRIEF DESCRIPTION OF THE DRAWINGS

FIG. 1 is a bar graph showing the estimated effects of nut consumption (10%, 12.2%, and 20% dietary energy from nuts respectively, based on a 2000-kcal diet.) on blood lipid and lipoprotein levels (HDL-C indicates high-density lipoprotein cholesterol; LDL-C, low-density lipoprotein cholesterol; TC, total cholesterol; and TG, triglycerides). Estimated using values from participants with triglyceride levels of at least 150 mg/dL (to convert triglyceride level to millimoles per liter, multiply by 0.0113). †Recommended by the US Food and Drug Administration; 12.2% is equivalent to 43 g/d (1.5 oz/d). At 20% of dietary energy from nuts (equivalent to 71 g [2.5 oz] for a 2000-kcal diet), blood lipid levels were reduced by 9.9 mg/dL (4.5% change) for TC and by 9.5 mg/dL (6.5% change) for LDL-C. At 12.2% of dietary energy from nuts (equivalent to 43 g [1.5 oz]), the amount of nut consumption recommended by the US Food and Drug Administration, 10 blood lipid levels were reduced by 7.1 mg/dL (3.2% change) for TC and by 7.2 mg/dL (4.9% change) for LDL-C.

FIG. 2A is a line graph showing the mean total point score (mean±SE) of visual field sensitivity (in decibel units or dB as measured with Humphrey Field Analyzer (HFA) 30-2 and 30/60-1 programs combined) among patients not on Vitamin A prior to entry over years (0, 1, 2, 3, and 4). In these subgroups, the difference between the 2 curves was larger during years 1 and 2 and smaller during years 3 and 4. Control group is signified by empty non-colored circles, DHA+A Group is represented by filled circles. Sample sizes for those not on vitamin A prior to entry were 28 for the DHA+A group and 34 for the control+A group; and for those on vitamin A prior to entry, 75 for the DHA+A group and 68 for the control+A group. Slopes of decline in total field sensitivity are shown for the DHA+A vs control+A groups for years 0 to 2 and 2 to 4 among those not on vitamin A prior to entry as seen in FIG. 2C. Limit lines depict standard error values.

FIG. 2B is a line graph showing the mean total point score (mean±SE) of visual field sensitivity (in decibel units or dB as measured with HFA 30-2 and 30/60-1 programs combined) among patients on Vitamin A prior to entry over years (0, 1, 2, 3, and 4). Among those on vitamin A prior to entry, the differences between the curves for the DHA+A vs control+A groups were not significantly different for either time period, although a slight divergence of the curves was noted particularly in years 3 and 4. Control group is signified by empty non-colored circles, DHA+A Group is represented by filled circles.

FIG. 2C is a bar graph showing the Total Point Score (mean±SE) Decline Among Patients Not on Vitamin A prior to entry (in decibel units or dB as measured with HFA) for years 0 to 2 and 2 to 4 when comparing the DHA+A (filled in bar) vs control+A groups (empty or non-colored bars). The rate of decline was significantly slower in the DHA+A vs control+A groups for years 0 to 2 (P=0.006), but was not significantly different for years 2 to 4 (P=0.57).

### DETAILED DESCRIPTION OF THE INVENTION

The present invention provides a novel composition of nut oils for use in maintaining and improving health. The present invention further provides a novel composition of nut oils for obtaining essential fatty acids that are required for normal body functioning. In an embodiment, the nut oil composition of the present invention provides walnut oil in an amount ranging between about 10% to about 35% by weight, almond oil in an amount ranging between about 10% to about 35% by weight, avocado oil in an amount ranging between about 10% to about 35% by weight and pistachio oil in an amount ranging between about 10% to about 35% by weight.

The walnut oil for use in the composition of the present invention is derived from the kernel of the walnut tree, Juglans regia. One method of extracting the oil from the walnut is to grind the walnut into a paste using often using a grinding machine to do so. Grinding machines for nuts are commercially available, and can be obtained, for example from Shandong Light M&E Co., Ltd (Shandong, China (Mainland)). The paste goes through a malaxation process, a slow stirring which encourages the oil in the paste to clump. To extract the oil, pressure is applied; forcing the oil out of the paste through the cold pressing in a climate-controlled environment where external temperatures are kept below 120 degrees F. Cold pressing machines are commercially available, and can be obtained, for example from Zhengzhou Dingsheng Machine Manufacturing Co., Ltd., Model No. 6YZ-260 (Henan, China (Mainland)). Walnut oil provides vitamin A, vitamin E, vitamin C, vitamin K, vitamin B1, vitamin B2, Pantothenic acid (vitamin B5), vitamin B6, niacin, folate and omega-3, omega-6, omega-7 and omega-9 fatty acids. Walnut oil is commercially available from Jedwards International, (Quincy, Mass.).

The almond oil for use in the composition of the present inventions is derived from the dried kernel of the almond tree, Prunus delcis and Prunus amara. The kernels, or fruit, of the almond tree contain almond oil. One method of extracting the oil from the almond is to grind the almond into a paste using often suing a grinding machine to do so. Grinding machines for nuts are commercially available, and can be obtained, for example from Shandong Light M&E Co., Ltd (Shandong, China (Mainland)). The paste goes through a malaxation process, a slow stirring which encourages the oil in the paste to clump. To extract the oil, pressure is applied; forcing the oil out of the paste through the cold pressing in a climate-controlled environment where external temperatures are kept below 120 degrees F. Cold pressing machines are commercially available, and can be obtained, for example from Zhengzhou Dingsheng Machine Manufacturing Co., Ltd., Model No. 6YZ-260 (Henan, China (Mainland)). Almond oil provides nutrients such as flavonoids, vitamin E, vitamin B1, vitamin B2, Pantothenic acid (vitamin B5), vitamin B6, niacin, folate, magnesium, manganese, copper and omega-3, omega-6, omega-7 and omega-9 fatty acids. Almond oil is commercially available from Jedwards International, Inc. (Quincy, Mass.).

The avocado oil for use in the composition of the present inventions is derived from the fleshy portion of the avocado fruit from the tree Persea americana. One method of extracting the oil from the avocado is to grind the avocado into a paste using often suing a grinding machine to do so. Grinding machines for nuts are commercially available, and can be obtained, for example from Shandong Light M&E Co., Ltd (Shandong, China (Mainland)). The paste goes through a malaxation process, a slow stirring which encourages the oil in the paste to clump. To extract the oil, pressure is applied; forcing the oil out of the paste through the cold pressing in a climate-controlled environment where external temperatures are kept below 120 degrees F. Cold pressing machines are commercially available, and can be obtained, for example from Zhengzhou Dingsheng Machine Manufacturing Co., Ltd., Model No. 6YZ-260 (Henan, China (Mainland)). Avocado oil contains vitamin A, vitamin E, amino acids, lecithin and omega-3, omega-6, omega-7 and omega-9 fatty acids. Avocado oil is commercially available from Jedwards International, Inc. (Quincy, Mass.).

The pistachio oil for use in the composition of the present invention is extracted from the fruit of Pistacia Vera, the pistachio nut. One method of extracting the oil from the pistachio is to grind the pistachio into a paste using often suing a grinding machine to do so. Grinding machines for nuts are commercially available, and can be obtained, for example from Shandong Light M&E Co., Ltd (Shandong, China (Mainland)). The paste goes through a malaxation process, a slow stirring which encourages the oil in the paste to clump. To extract the oil, pressure is applied; forcing the oil out of the paste through the cold pressing in a climate-controlled environment where external temperatures are kept below 120 degrees F. Cold pressing machines are commercially available, and can be obtained, for example from Zhengzhou Dingsheng Machine Manufacturing Co., Ltd., Model No. 6YZ-260 (Henan, China (Mainland)). Pistachio oil contains vitamin C, vitamin B1, vitamin B2, Pantothenic acid (vitamin B5), vitamin B6, vitamin A, vitamin E and vitamin K, niacin, folate and omega-3, omega-6, omega-7 and omega-9 fatty acids. Pistachio oil is commercially available from Jedwards International, Inc. (Quincy, Mass.).

### Modes and Manner of Administration, Dosages

The composition of nut oils used in the present invention can be administered with or without a carrier. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrolidone, calcium carbonate, carbohydrates, such as lactose, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17^{th} Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include potassium sorbate, sodium metabisulfite, methyl paraben, propyl paraben, thimerosal, etc. The compositions can also be combined where desired with other active substances. A carrier (e.g., a pharmaceutically acceptable carrier) is preferred, but not necessary to administer the compound.

In a preferred embodiment, the nut oil composition of the present invention is administered in the form of a softgel capsule. The softgel capsule can be made from aqueous solutions of gelling agents such as animal protein, namely, gelatin and/or plant polysaccharides or their derivatives like carrageenans and modified forms of starch and cellulose. Other ingredients can be added to the gelling agent solution like plasticizers such as glycerin and/or sorbitol to decrease the capsule's hardness, coloring agents, preservatives, disintegrants, lubricants and surface treatment. The encapsulation process begins when molten gel is pumped to the machine and two thin ribbons of gelatin are formed. These ribbons then pass over a series of rollers and are continuously fed between two rotating die cylinders that determine the size and shape of the capsules, which form the two halves of a capsule. A fill injector is used to fill the appropriate volume of material to be encapsulated. As the die assembly rotates, the filled capsule halves are then sealed together by the application of heat and pressure and are then ejected. Following encapsulation, the capsules undergo drying in tumblers containing lint-free towels and large volumes of forced air. The capsules are then placed into low-humidity drying rooms, where the soft gels are dried to remove any excess moisture. Examples of manufacturers of softgel encapsulation include Pharmland Technologies (Ontario, Canada) or Zhejiang Jiangnan Pharmaceutical Machinery Co., Ltd. (Zhejiang, China).

The composition of nut oils can be a liquid solution, suspension, emulsion, tablet, pill, capsule, sustained release formulation, or powder. The method of administration can dictate how the composition will be formulated. Oral formulation can include standard carriers such as pharmaceutical grades of mannitol, lactose, starch, magnesium stearate, sodium saccharine, cellulose, magnesium carbonate, etc. For example, the composition can be formulated as a suppository, with traditional binders and carriers such as triglycerides.

The composition of nut oils used in the invention can be administered orally, intravenously, parenterally, intramuscular, subcutaneously, nasally, topically, by inhalation, by implant, by injection, or by suppository. The composition can be administered in a single dose or in more than one dose over a period of time to confer the desired effect. In one embodiment, the composition of nut oils, can be administered orally in an amount between about 0.25 g to about 10 g/daily, and in an embodiment between about 0.5 g and about 3 g/daily (e.g., about 0.5 g, 1 g, 1.5 g, 2 g, 2.5 g, or 3 g/daily).

In an embodiment, the actual effective amounts of a composition can vary according to the specific composition being utilized, the mode of administration and the age, weight and condition of the individual. For example, as used herein, an effective amount of the composition of nut oils is an amount which maintains and improves health. Dosages for a particular individual can be determined by one of ordinary skill in the art using conventional considerations, (e.g. by means of an appropriate, conventional pharmacological protocol).

For enteral or mucosal application (including via oral and nasal mucosa), particularly suitable are tablets, liquids, drops, suppositories or capsules. A syrup, elixir or the like can be used wherein a sweetened vehicle is employed. Liposomes, microspheres, and microcapsules are available and can be used.

For parenteral application, particularly suitable are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like. Ampules are convenient unit dosages.

The administration of combinations of the components of the nut oil composition described herein can occur simultaneously or sequentially in time. Additionally, the nut oil composition of the present invention can be administered with an additional compound (e.g., vitamin, mineral, supplement or any combination thereof) simultaneously or sequentially in time. The additional compound can be administered before, after or at the same time as the nut oil composition. Thus, the term “co-administration” is used herein to mean that the nut oil composition and the additional compound will be administered at times to maintain or improve health. The methods of the present invention are not limited to the sequence in which the nut oil composition and additional compound are administered, so long as the nut oil composition and additional compound are administered close enough in time to produce the desired effect.

Referring to Table 1 below shows the fatty acid composition of nut oils, specifically, walnut oil, almond oil, avocado oil and pistachio oil.

TABLE 1

| | | | | | | Alpha | | | | |
|---|---|---|---|---|---|---|---|---|---|---|
| | | | | Oleic | Linolenic | Linolenic | Palmitoleic | Hepta- | | |
| | Fat | Palmitic | Stearic | Acid | Acid | Acid | Acid | Decanoic | Arachidic | Eicosenoic |
| | Content | Acid | Acid | (Omega-9) | (Omega-6) | (Omega-3) | (Omega-7) | Acid | Acid | Acid |
| | in seed | (%) | (%) | (%) | (%) | (%) | (%) | (%) | (%) | (%) |
| Oil or fat | (%) | C16:0 | C18:0 | C18:1 | C18:2 | C18:3 | C16:1 | C17:0 | C20:0 | C20:1 |
| Walnut | 60 | 6.7-8.0 | 2.3-2.6 | 15.0-19.0 | 58.0-61.0 | 11.0-13.5 | 0-1.0 | 0-1.0 | 0-1.0 | 0-1.0 |
| Oil | | | | | | | | | | |
| Almond | 54.2 | 3.0-9.0 | 0-3.0 | 60.0-75.0 | 20.0-30.0 | 0-0.4 | 0-0.7 | 0 | 0 | 0 |
| Oil | | | | | | | | | | |
| Avocado | 12 | 5.0-15.0 | 0-3.0 | 59.0-74.0 | 10.0-20.0 | 0-3.0 | 0-5.0 | 0 | 0 | 0 |
| Oil | | | | | | | | | | |
| Pistachio | 53.7 | 10.0-13.0 | 1.0-3.0 | 50.0-55.0 | 30.0-35.0 | 0-1.0 | 1.0-3.0 | 0 | 0 | 0 |
| Oil | | | | | | | | | | |
| Total | | 6.2-11.3 | 0.8-3.0 | 46.0-56.0 | 30.0-37.0 | 3.0-4.5 | .25-2.4 | .25 | .25 | .25 |
| Range | | | | | | | | | | |

The nut oil composition of the present invention provides the following fatty acids selected from the group consisting of palmitic acid (C16:0), palmitoleic acid (C16:1), heptadecanoic acid (C17:1), stearic acid (C18:0), arachidic acid (C20:0), eicosenoic acid (C20:1), linoleic acid (C18:2), linolenic acid (C18:3), oleic acid (C18:1) or any combination thereof. In an embodiment, the nut oil composition of the present invention provides palmitic acid in an amount ranging between about 5 to about 20% by weight (e.g., about 6% and about 12%, or 7%, 8%, 9%, 10%, 11% or 12%), stearic acid in an amount ranging between about 0.5% to about 10% by weight (e.g., about 0.8% and about 3%, or in particular, 1%, 2%, or 3%), oleic acid in an amount ranging between about 40% and about 60% by weight (e.g., about 46% and about 56%, or in particular, 47%, 48%, 49%, 50%, 55% or 56%), linoleic acid in an amount ranging between about 25% to about 45% by weight (e.g., about 30.0% and about 37.0%, or in particular, 34%, 35%, 36%, or 37%), alpha linolenic acid in an amount ranging between about 2% and about 10% by weight (e.g., about 3.0% and about 4.4% or in particular 3.5%, 4%, or 4.4%), palmitoleic acid in an amount ranging between about 0.1% and about 5% (e.g., about 0.25% and about 1.2% or in particular 0.50%, 1%, or 1.2%), heptadecanoic acid in an amount ranging between about 0.1% and about 0.25% by weight (e.g., 0.12%, 0.15%, 0.20%, or 0.25%), arachidic acid in an amount ranging between about 0.1% and about 0.25% by weight (e.g., 0.12%, 0.15%, 0.20%, or 0.25%) and eicosenoic acid in an amount between about 0.1% and about 0.25% by weight (e.g., 0.12%, 0.15%, 0.20%, or 0.25%).

In another embodiment, the nut oil composition of the present invention includes polyunsaturated fatty acids, monounsaturated fatty acids, saturated fatty acids including omega-3, omega-6, omega-7 and omega-9 fatty acids. In an aspect, the nut oil composition of the present invention provides an amount of omega-3 in the range of about 2% and about 10% total lipids composition (e.g., about 3.0% and about 4.4% or in particular 3.5%, 4%, or 4.4%), an amount of the omega-6 ranging between about 25% to about 45% total lipids in composition (e.g., about 30.0% and about 37.0%, or in particular, 34%, 35%, 36%, or 37%), an amount of the omega-7 in the range between about 0.1% and about 5% total lipids in composition (e.g., about 0.25% and about 1.2% or in particular 0.50%, 1%, or 1.2%), and an amount of the omega-9 in a range between about 40% and about 60% total lipids in composition (e.g., about 46% and about 56%, or in particular, 47%, 48%, 49%, 50%, 55% or 56%),

In yet another embodiment, the nut oil composition of the present invention provides walnut oil in an amount ranging between about 100 mg/mL and about 350 mg/mL, almond oil in an amount ranging between about 100 mg/mL and about 350 mg/mL, avocado oil in an amount ranging between about 100 mg/mL and about 350 mg/mL and pistachio oil in an amount ranging between about 100 mg/mL and about 350 mg/mL. In an aspect, the nut oil composition of the present invention provides walnut oil, almond oil, avocado oil and pistachio oil in an amount of 250 mg/mL or 0.25 g/L. In yet another embodiment, the nut oil composition of the present invention provides walnut oil, almond oil, avocado oil and pistachio oil as 1:1:1:1 ratio.

In an embodiment, the nut oil composition of the present invention further comprises antioxidants, vitamins, minerals, phytonutrients, fatty acids or any combination thereof. Antioxidants are widely used as ingredients in dietary supplements for maintaining health and preventing diseases such as cancer and coronary heart disease. Dietary and nutritional supplements, often containing vitamins, are used to ensure that adequate amounts of nutrients are obtained on a daily basis, if optimal amounts of the nutrients cannot be obtained through a varied diet. The health benefits of vitamins include their ability to prevent and treat various diseases including heart problems, high cholesterol levels, eye disorders, and skin disorders. Walnuts, almonds, avocados and pistachio nuts all naturally contain vitamin E.

Phytonutrients are natural bioactive compounds found in fruits and vegetables that work together with vitamins and minerals to promote good health. In an embodiment, the nut oil composition of the present invention further includes vitamin E in an amount ranging between 10% and about 15% by weight (e.g., 11%, 12%, 13%, 14%).

The present invention further provides methods for maintaining and improving health. In particular, the present invention provides methods of treating or reducing abnormal total cholesterol levels, abnormal blood lipid levels or both in an individual, by selecting an individual characterized by a high total cholesterol level, a high blood lipid levels or both; and administering to the individual an effective amount of the nut oil composition, as described herein, to decrease total cholesterol levels, improve blood lipid level or both. In an embodiment, the methods include administering the nut oil composition described herein e.g., one that includes a walnut oil in an amount ranging between about 10% and about 35% by weight, an almond oil in an amount ranging between about 10% and about 35% by weight, an avocado oil in an amount ranging between about 10% to about 35% by weight and a pistachio oil in an amount ranging between about 10% to about 35% by weight. In an aspect, administration of the nut oil composition to an individual having a high total cholesterol level, a high blood lipid level or both, results in a decrease of the total cholesterol level, the blood lipid level or both by 5% (e.g., 10%, 15%, 20%, 25%, 30%, 35%, 40%, 50%, 60%, 70%, 80%, or 90%).

In one aspect, the present invention involves assessing the total cholesterol levels, blood lipid levels or both in individuals, to determine if either or both levels are elevated. Individuals with elevated total cholesterol levels and/or blood lipid levels can be at risk for vascular events. Individuals can have other risk factors for vascular events, as further described herein. Administration of the nut oil composition of the present invention decreases total cholesterol levels and/or blood lipid levels, as compared to level(s) prior to administration of the nut oil composition.

A blood lipid levels or lipid profile is made up of a number of items including, but not limited to, High Density Lipoprotein-Cholesterol (HDL-C), Low Density Lipoprotein-Cholesterol (LDL-C), triglycerides, and total Cholesterol (Total-C). Lipoproteins are complexes which contain both a lipid and protein. Most of the lipids in plasma are present as lipoproteins and are transported as such. Lipoproteins are characterized by their flotation constants (e.g., densities). Various classes of lipoproteins exist and include HDLs and LDLs. LDLs are particularly rich in cholesterol esters.

Levels of cholesterol that are considered normal can vary and depend on factors such as the individual's health history, the number of risk factors the individual has, etc. The National Cholesterol Education Program (NCEP) Guidelines state that a Total-C of greater than or equal to 200 mg/dL, and a LDL-C of greater than or equal to 130 mg/dL are considered borderline high. In one embodiment, the present invention relates to administering a (e.g., one or more) nut oil composition to an individual with Total-C and/or LDL-C level(s) that are considered to be high or borderline high (e.g., individuals with levels on the higher side of a normal ranges). For example, a normal range of Total-C is considered to be between about 60 mg/dL and about 199 mg/dL, and LDL-C is between about 85 mg/dL and about 129 mg/dL. As such, the present invention embodies administering the nut oil composition to an individual having a Total-C of greater than about 200 mg/dL (e.g., between about 200 mg/dL and about 350 mg/dL) and/or an LDL-C of greater than about 130 mg/dL (e.g., between about 130 mg/dL and 280 mg/dL). Additionally, the present invention embodies administering the nut oil composition to an individual with high triglyceride levels, e.g., administering the nut oil composition to an individual with a triglyceride level of greater than 150 mg/dL (e.g., between about 150 mg/dL and about 300 mg/dL).

One embodiment of the invention includes administering the nut oil composition to an individual having a Total-C and/or a LDL-C level in the upper end of the normal range. For example, an individual having a LDL-C level in the range of about 100 mg/dL and about 129 mg/dL is considered to be in the normal range, but not optimal. Similarly, an individual with a Total-C in a range from about 170 mg/dL to about 199 mg/dL are also considered to be normal, but not optimal. As such, individuals at risk for a vascular event, as described herein, and have levels of LDL-C or Total-C in the upper portion of the normal range can benefit from the invention. Hence, in one embodiment, the present invention relates to administering the nut oil composition to an individual with a LDL-C level of between about 100 mg/dL and about 129 mg/dL, and/or a Total-C level of about 170 mg/dL and about 199 mg/dL. Similarly, triglyceride levels that are on the upper end of the normal range include levels between about 100 mg/dL and about 149 mg/dL. The invention therefore embodies administering the nut oil composition to an individual that has a triglyceride level between about 100 mg/dL and about 149 mg/dL, wherein the triglyceride level is reduced.

Lipoproteins levels and triglyceride levels are measured and assessed using routine methods known in the art. Commercially available kits and assays can be used to evaluate the levels of Total-C, HDL-C, LDL-C and triglycerides. A reduction in the total cholesterol levels, blood lipid levels or both refers to a decrease in one or both, as compared to levels prior to administration or as compared to a control, as further described herein. Levels of one or both can be decreased or reduced by at least about 5% (e.g., 10%, 15%, 20%, 25%, 30%, 35%, 40%, 50%, 60%, 70%, 80%, or 90%), as compared to the level just prior to administration. Accordingly, one can measure the presence, absence or level of one or both, and compare the result against a control. For example, one can obtain a suitable sample and compare the level of one or both from previous time points (e.g., prior to administration of the nut oil composition or during the onset of a vascular event, disease or disorder). The total cholesterol level and/or one or more blood lipid levels decrease after administration of the nut oil composition, as compared to the level during the onset of the vascular event. Total cholesterol levels and/or blood lipid levels can be assessed and compared to a standard or control obtained from normal individuals. In one example, total cholesterol levels and/or blood lipid levels can be assessed in a population of healthy individuals or individuals who have not had a vascular event, disease or disorder. Such levels are referred to as a “negative control.” Conversely, total cholesterol levels and/or blood lipid levels can also be obtained from a pool of individuals who are known to have high cholesterol levels or are undergoing a vascular event, disease or disorder, e.g., a “positive control.” After administration of the nut oil composition, the level of total cholesterol, blood lipid or both decreases; the level(s) get closer to the level of the negative control, and farther from the positive control. The total cholesterol levels and/or blood lipid levels decrease as compared to the levels during the onset of the vascular event, disease or disorder. Hence, the methods of the present invention include reducing total cholesterol levels and/or blood lipid levels, with administration of the nut oil composition, wherein total cholesterol levels, blood lipid levels or both are reduced or decreased, as compared to those levels prior to the administration of the nut oil composition.

In another embodiment, an effective amount of the nut oil composition can be administered to prevent total cholesterol levels and/or blood lipid levels from increasing, or lessen total cholesterol levels and/or blood lipid levels which would otherwise remain elevated without nut oil composition administration. For example, an individual who is a risk for elevated cholesterol levels or who have other associated risk factors such a vascular event, disease or condition can take the nut oil composition on a daily basis (or every other day), to prevent total cholesterol levels and/or blood lipid levels from increasing as compared to a control or baseline. Baseline levels can be obtained prior to and/or during the course of administration of the nut oil composition. Total cholesterol levels and/or blood lipid levels can stay the same, or can even decrease. Similarly, total cholesterol levels and/or blood lipid levels can be compared to a negative or positive control, wherein upon administration of the nut oil composition, the levels are closer to the negative control, than the positive control.

The present invention pertains to methods for preventing or treating an individual at risk for or having a (e.g., one or more) vascular event, disease or disorder. Administration of the nut oil composition described herein reduce the severity of or prevent vascular events. Prevention of a vascular event, condition, disease or disorder (e.g., thrombotic event, condition, disease or disorder) refers to delaying or suppressing the onset of the vascular disorder, or one or more of its symptoms. To treat an individual at risk for a vascular disorder means to alleviate, ameliorate or reduce the severity of one or more of its symptoms.

Two phases of thrombotic events e.g., cardiovascular and/or cerebrovascular events, can exist: an ischemic stage and a necrotic stage. A patient can suffer from ischemia in which a decrease of blood flow can occur. This decrease in blood flow causes a decrease in tissue oxygenation. After prolonged ischemia, the tissue can undergo necrosis which is death of the tissue. Therefore, patients who are at risk for a vascular event can exhibit elevated levels of ischemic markers and/or necrosis markers.

The methods include selecting an individual at risk for a vascular event and administering the nut oil composition described herein. An individual at risk for a vascular disorder refers to an individual with a history of vascular disease, an individual experiencing at least one symptom of the disorder, an individual having known risk factors (e.g., gender, weight) associated with or caused by the vascular disorder, an individual undergoing a vascular procedure, or an individual who has tested positive for a vascular condition using a diagnostic test (e.g., electrocardiogram, cardiac catheterization, stress test, ultrasound techniques, laboratory tests).

As described above, an embodiment of the invention includes preventing, or treating one or more symptoms exhibited by individuals having or at risk for a vascular event. Individuals at risk for vascular events are those that manifest at least one symptom indicative of a vascular disorder/event. Symptoms that are indicative of a coronary-related vascular event, for example, include chest pain, abnormal electrocardiograms, elevated levels of ischemic markers, necrosis markers, or thrombin/fibrin generation markers. Such markers include, but are not limited to, Creatine Kinase with Muscle and/or Brain subunits (CKMB), D-Dimer, F1.2, thrombin anti-thrombin (TAT), soluble fibrin monomer (SFM), fibrin peptide A (FPA), myoglobin, thrombin precursor protein (TPP), platelet monocyte aggregate (PMA) and troponin. Individuals who are at risk also include those having a history of a vascular event (e.g. disorder), including Coronary Heart Disease (CHD), stroke, or Transient Ischemic Attacks (TIAs). A history of CHD can include, for example, a history of MI, coronary revascularization procedure, angina with ischemic changes, or a positive coronary angiogram (e.g., showing greater than about 50% stenosis of at least one major coronary artery).

Vascular events, diseases or disorders include cardiovascular diseases (e.g., coronary heart disease, myocardial infarction, angina or a disease in which a narrowing of a blood vessel occurs in at least one major artery), cerebrovascular diseases (e.g., stroke or transient ischemic attacks), vascular procedures (e.g., thrombotic re-occlusion subsequent to a coronary intervention procedure, heart or vascular surgery) or any other thrombotic event (e.g., pulmonary embolism, deep vein thrombosis or peripheral vascular thrombosis). Vascular disorders also include Syndrome X, which is a disease that is associated with unidentified chest pain.

The present invention also relates to methods for reducing the occurrence or severity of a vascular disorder in a patient who is at risk for such a disorder or has the disorder. Reducing the occurrence of a vascular disorder (e.g., a cardiovascular and/or cerebrovascular disorder) refers to reducing the probability that a patient will develop the disorder, or delaying the onset of the disorder. Reducing the severity of a vascular disorder refers to a reduction in the degree of at least one symptom of the disorder. In an embodiment, nut oil administration decreases or reduces symptoms associated with a vascular event by at least about 5% (e.g., 10%, 15%, 20%, 25%, 30%, 35%, 40%, 50%, 60%, 70%, 80%, or 90%), as compared to the level just prior to administration. The present invention embodies methods for preventing or delaying the onset of a vascular disorder in an individual by administering the nut oil composition of the present invention.

Administering the nut oil formation described herein can result in a reduction of recurrent vascular events (e.g., cardiovascular and/or cerebrovascular events). Use of nut oil administration described herein results in a reduction of at least about 5%, (e.g., 10%, 15%, 20%, 25%) in the number of recurrent heart attacks, cardiac deaths and/or strokes.

A vascular disorder is an event, disease or disorder that involves a thrombosis (e.g., a thrombotic event) or a narrowing of a blood vessel. A vascular disorder/event occurs, for example, when a clot forms and lodges within a blood vessel. The blockage can fully block or partially block the blood vessel causing a vascular disorder. Thrombin generation refers to the activation, expression or up-regulation of thrombin, which is involved in clot formation and an inductor of platelet activation. The amount of thrombin generation can be measured or assessed by certain markers known in the art. Examples of such thrombin generation markers include fibrinopeptide A, prothrombin fragments 1 +2, and thrombin-antithrombin-III complexes.

The present invention further relates to methods of reducing retinal degeneration in an individual with retinitis pigmentosa and methods of reducing the decline in visual field sensitivity in an individual with retinitis pigmentosa.

Retinitis pigmentosa refers to an eye disease in which there is damage to the retina. Specifically, the disorder is caused by genetic defects that result in damage to the rods cells and/or cone cells. The retina is the layer of tissue at the back of the inner eye that converts light images to nerve signals and sends them to the brain. Retinitis pigmentosa is characterized by the presence of dark deposits in the retina.

An individual at risk for retinitis pigmentosa refers to an individual with a history of the condition, an individual experiencing at least one symptom of the condition, an individual having known risk factors (e.g., genetic defects, family history of the disorder, Usher syndrome) associated with or caused by the condition, or an individual who has tested positive for the condition using a diagnostic test (e.g., visual field testing, electroretinogram (ERG), fluorescein angiography, measurement of the electrical activity in the retina, refraction test, visual acuity and the like). Symptoms associated with retinitis pigmentosa include e.g., decreased vision at night or in low light, loss of peripheral vision (e.g. side vision causing tunnel vision) and/or loss of central vision.

Reducing the severity of this condition refers to a reduction in the degree of at least one symptom of the condition as characterized by one or more test results indicative of the condition. In an aspect, the present invention involves assessing one or more symptoms indicative of the condition to determine if the symptom is worsened, as compared to the symptom in an individual without the condition. Improving test results refers to levels or results that get closer to the normal range for that test, as compared to those prior to administering the nut oil formulation described herein. In an embodiment, administration of the nut oil formation described herein reduces one or more of symptoms of the disease, as compared to extent or degree of the symptom prior to administration of the nut oil formulation or to a control (e.g., an individual with the disorder and not taking the nut oil composition of the present invention). Controls and assessment of various symptoms are known in the art. The present invention includes symptoms or markers that are discovered in the future that are indicative of retinitis pigmentosa.

In an embodiment, administering the nut oil formation described herein to an individual having retinitis pigmentosa, results in a reduction of the retinal degeneration associated with the condition by at least about 5% (e.g., 10%, 15%, 20%, 25%, 30%, 35%, 40%, 50%, 60%, 70%, 80%, or 90%) as compared to the level prior to administration or prevents retinal degeneration from increasing, as compared to levels prior to administration.

In yet another embodiment, administering the nut oil formation described herein to an individual having retinitis pigmentosa, results in a reduction of the decline in visual field sensitivity associated with the condition by at least about 5% (e.g., 10%, 15%, 20%, 25%, 30%, 35%, 40%, 50%, 60%, 70%, 80%, or 90%) as compared to the level prior to administration, or prevents visual field sensitivity from increasing, as compared to levels prior to administration.

### EXEMPLIFICATION

### Example 1

In a pooled analysis of 25 individual nut consumption trials (including walnuts, almonds and pistachios) conducted in seven countries among 583 men and women with normolipididemia and hypercholesterolemia who were not taking lipid-lowering medications, the effects of nut consumption and the potential interactions were assessed.

Of the 25 studies in the pooled analysis, 16 used a cross over design, 7 used a consecutive design and 2 used a parallel design. Sample size ranged from 10 to 49 subjects (median, 20 subjects) and age ranged from 19 to 86 years (mean age, 46 years). 21 out of 25 studies included both sexes and there were 307 men and 276 women. Subjects in 9 studies had hypercholesterolemia of 236-259 mg/dL for total cholesterol concentration (TC) and 154-178 mg/dL for low-density lipoprotein cholesterol concentration (LDL-C). Subjects in 16 studies had normocholesterolemia of 125-222 mf/dL for TC and 67-142 mg/dL for LDL-C. Across studies, subjects BMIs ranged from 17-49 (mean, 27). Daily nut consumption ranged from 23-132 g (mean, 67 g) or 0.8 to 4.8 oz/d (mean, 2.4 ox/d).

Referring to Table 2 below shows estimated changes in blood lipid and lipoprotein levels among subjects consuming nut diets vs. control diets. Compared with control diets, nut diets reduced TC, LDL-C, ratio of LDL-C to high-density lipoprotein cholesterol concentration (HDL-C) and ratio of TC to HDL-C (p<0.001 for all) where p=the difference between nut diet and control diet. The effects of nut consumption on blood lipid levels were similar in men and women and across all age groups.

TABLE 2
Estimated Changes in Blood Lipid and Lipoprotein Levels among
Subjects Consuming Nut Diets vs. Control Diets

| | Mean Change | | |
|---|---|---|---|
| Variable | (95% Confidence Interval)^{a} | % Change | P Value^{b} |
| Concentration, | | | |
| mg/dL | | | |
| TC | −10. (−14.1 to −7.8) | −5.1 | <.001 |
| LDL-C | −10.2 (−13.1 to −7.4) | −7.4 | <.001 |
| HDL-C | 0.09 (−1.00 to 1.19) | 0.2 | .88 |
| Ratio | | | |
| LDL-C/HDL-C | −0.2 (−0.3 to −0.1) | −8.3 | <.001 |
| TC/HDL-C | −0.2 (−0.3 to −0.1) | −5.6 | <.001 |
| Triglyceride level, | −3.1 (−7.2 to 1.2) | −2.8 | .15 |
| mg/dL | | | |
| <150 | 0.7 (−3.2 to 4.7) | 0.7 | .74 |
| ≧150 | −20.6 (−30.7 to −9.9) | −10.2 | <..05 |

The estimated cholesterol lowering effects of nut consumption were greater for subjects with higher baseline LDL-C.

Referring to Table 3 below shows the estimated changes in blood lipid and lipoprotein levels by baseline LDL-C concentration and by baseline BMI among subjects consuming nut diets vs. control diets. Responses differed between subjects with baseline LDL-C of less than 130 mg/dL vs. greater than 160 mg/dL (mean decrease, 12.5 mg/dL for TC and 14.9 mg/dL for LDL-C). Similarly, there was a differential cholesterol lowering effect of nut consumption depending on baseline BMI, with greater response among subjects with having lower BMI. A significant nut diet with X BMI interaction was found for a ration of LDL-C to HDL-C (p=0.02) and for ratio of TC to HDL-C (p=0.02).

TABLE 3
Estimated Changes in Blood Lipid and Lipoprotein Levels by baseline LDL-C Concentration and
by Baseline BMI among Subjects Consuming Nut Diets vs. Control Diets

| | | TC | LDL-C | | | Triglyceride |
|---|---|---|---|---|---|---|
| | | Concentration, | Concentration, | LDL-C/ | TC/ | Level, |
| Variable | No. | mg/dL | mg/dL | HDL-C | HDL-C | mg/dL^{a} |
| LDL-C | | | | | | |
| concentra- | | | | | | |
| tion, mg/dL | | | | | | |
| <130 | 262 | −5.0 (−9.2 to −0.9)^{b} | −3.5 (−7.5 to 0.5) | −0.11 (−0.19 to −0.02)^{b} | −0.14 (−0.24 to −0.04)^{b} | −2.0 (−6.5 to 2.8) |
| 130-160 | 125 | −11.0 (−15.5 to 31 6.6)^{c} | −9.9 (−14.2 to −5.6)^{c} | −0.26 (−0.38 to −0.13)^{b} | −0.28 (−0.41 to −0.15)^{c} | −8.5 (−14.7 to −1.3)^{b} |
| >160 | 195 | −17.5 (−22.0 to −13.0)^{c} | −18.4 (−22.7 to −14.1)^{c} | −0.38 (−0.52 to −0.24)^{c} | −0.35 (−0.48 to −0.20)^{c} | −0.6 (−7.1 to 6.3) |
| BMI | | | | | | |
| <25 | 244 | −12.0 (−15.9 to −8.1)^{c} | −11.9 (−15.4 to −8.4)^{c} | −0.24 (−0.32 to −0.16)^{c} | −0.24 (−0.33 to −0.15)^{c} | −5.8 (−9.8 to −1.6)^{b} |
| 25-30 | 181 | −10.5 (−14.4 to −6.6)^{c} | −9.2 (−12.8 to −5.7)^{c} | −0.14 (−0.23 to −0.04)^{b} | −0.15 (−0.25 to −0.04)^{b} | −0.6 (−6.0 to 5.0) |
| >30 | 82 | −8.9 (−13.7 to −4.1)^{b} | −6.8 (−11.2 to −2.4)^{b} | −0.10 (−0.21 to 0.02) | −0.12 (−0.25 to 0.01) | −1.6 (−9.2 to 6.4) |

Accordingly, the cholesterol lowering effects of nut consumption are dose related and are more pronounced in subjects with higher baseline LDL-C or lower BMI. Specifically, a mean daily consumption of 67 g (2.4 oz) of nuts resulted in estimated mean reductions of 10.9 mg/dL (5.1% change) in TC, 10.2 mg/dL (7.4% change) in LDL-C, 0.22 (8.3% change) in ratio of LDl-C to HDL-C and 0.24 (5.6% change) in ratio of TC to HDL-C.

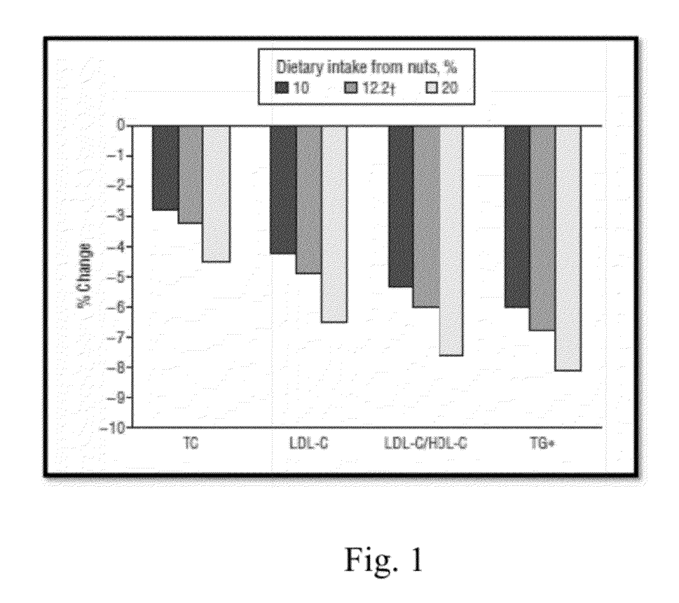

Referring to FIG. 1, shows that the estimated effects of nut consumption on blood lipid levels were dose related. At 20% of dietary energy from nuts, equivalent to 71 g or 2.5 oz for a 2000-kcal diet, blood lipid levels were reduced by 9.9 mg/dL (4.5% change) for TC and by 9.5 mg/dL (6.5% change) for LDL-C. At 12.2% of dietary energy from nuts, equivalent to 43 g or 1.5 oz, blood lipid levels were reduced by 7.1 mg/dL (3.2% change) for TC and by 7.2 mg/dL (4.9% change) for LDL-C. At 10% of dietary energy from nuts, equivalent to 35 g or 1.2 oz, blood lipid levels were reduced by 6.1 mg/dL (2.8% change) for TC and 6.2 mg/dL (4.2% change) for LDL-C.

In summary, cholesterol lowering effects of nut consumption are dose related and are more pronounced in subjects with higher baseline LDL-C or lower BMI. Nut consumption also lowered triglyceride levels in subjects with hypertriglyceridemia. Sabate, Joan “Nut Consumption and Blood Lipid Levels” Arch Intern Med, Vol 170 (No. 9) May 2010.

### Example 2

For patients with retinitis pigmentosa beginning vitamin A therapy, addition of docosahexaenoic acid (DHA) slowed the course of disease. Among patients on Vitamin A for at least 2 years, a diet rich omega-3 fatty acids slowed the decline in visual field sensitivity.

208 patients with retinitis pigmentosa, aged 18 to 55 years, were randomly assigned 1200 mg of docosahexanoic acid (DHA) plus 15000 IU/d of vitamin A given as retinyl palmitate (DHA+A group) or control fatty acid plus 1500 IU/d of vitamin A (control+A group) and followed up over four years. 70% of patients in each group were taking vitamin A, 15000 IU/d, prior to entry. Rates of decline in ocular function in the DHA+A vs control+A groups among the subgroups defined by use or nonuse of vitamin A prior to entry were compared. Also, it was determined whether decline in ocular function was related to red blood cell phosphatidylethanolamine docosaheaxanoic acid level, dietary omega-3 fatty acid intake or duration of vitamin A use.

Among patients not taking vitamin A prior to entry, those in the DHA+A group had a slower decline in field sensitivity and electroretinogram amplitude than those is the control+A group over the first two years (P=0.01 and P=0.3, respectively); these differences were not observed in years 3 and 4 of follow up or among patients taking vitamin A prior to entry. Additionally, in the entire control+A group, dietary omega-3 fatty acid intake was inversely related to the loss of total field sensitivity over 4 years (intake, <0.20 vs ≧0.20 g/d; P=0.02).

Referring to Table 4 below shows the mean annual rates of decline of central and total visual field sensitivity, 30-Hz ERG (Electroretinogram) amplitude, and Early Treatment Diabetic Retinopathy Study (ETDRS) acuity over a 4-year interval among the 208 patients in the DHA+A vs control+A groups for those on and not on vitamin A prior to entry.

TABLE 4
Annual Rate of Decline for Measures of Ocular Function by Treatment Group
and Vitamin A Status Prior to Entry over a 4-year Interval.

| On Vitamin A Prior to Entry | | | | Not on Vitamin A Prior to Entry | | | |
|---|---|---|---|---|---|---|---|
| | | | P | | | | P Value |
| | DHA + A | Control + A | Value† | DHA + A | Control + A | P Value† | Interaction† |
| HFA 30-2 | 39.41 ± 3.76 | 30.26 ± 3.92 | .09 | 30.7 ± 6.48 | 52.5 ± 5.99 | .01 | .002 |
| field, dB/y | (74) | (68) | | (29) | (34) | | |
| HFA total | 61.01 ± 5.17 | 48.13 ± 5.39 | .08 | 47.16 ± 10.56 | 82.49 ± 9.58 | .01 | .001 |
| field, dB/y‡ | (74) | (68) | | (28) | (34) | | |
| 30-Hz ERG, | 0.11 ± 0.01 | 0.10 ± 0.01 | .34 | 0.08 ± 0.02 | 0.14 ± 0.02 | .03 | .02 |
| logₑ | (75) | (67) | | (26) | (33) | | |
| % Decline | 10.57 | 9.23 | | 8.05 | 12.99 | | |
| per year§ | | | | | | | |
| ETDRS | 0.67 ± 0.13 | 0.68 ± 0.14 | .96 | 0.82 ± 0.26 | 0.69 ± 0.24 | .70 | .62 |
| visual acuity, | (75) | (68) | | (29) | (34) | | |
| letters per | | | | | | | |
| year | | | | | | | |

*Unless otherwise indicated, data are expressed as mean ± SE (number of patients sampled). Patients received either 1200 mg/d of docosahaxaenoic acid plus 15 000 IU/d of vitamin A (DHA + A) or control capsules plus 15 000 IU/d of vitamin A (control + A).
†Calculated from PROC MIXED (SAS Institute Inc., Cary, NC) analysis comparing rates of decline in both treatment groups.
‡Total field sensitivity consists of 30-2 and 30/60-1 total point scores combined when both are available.
§Derived from 100 × [1 − exp(mean log change)].

There were significant statistical interactions of treatment effects according to vitamin A supplement use prior to entry for visual field sensitivity and 30-Hz ERG amplitude, suggesting that treatment group effects were different for those on vs not on vitamin A prior to entry. For those on vitamin A prior to entry, the mean annual rates of decline of central and total field sensitivity and 30-Hz ERG amplitude were not significantly different between the DHA+A group and the control+A groups. For those not on vitamin A prior to entry, mean rates of decline of central and total field sensitivity and 30-Hz ERG amplitude were significantly lower in the DHA+A vs control+A groups. No significant statistical interaction effect was noted for ETDRS acuity, and no significant differences in rates of ETDRS acuity decline were noted in subgroup comparisons. Berson, Eliot L., et al., “Further Evaluation of Docosahexaenoic Acid in Patients with Retinitis Pigmentosa Receiving Vitamin A Treatment” Arch Ophthalmol, Vol 122 September 2004.

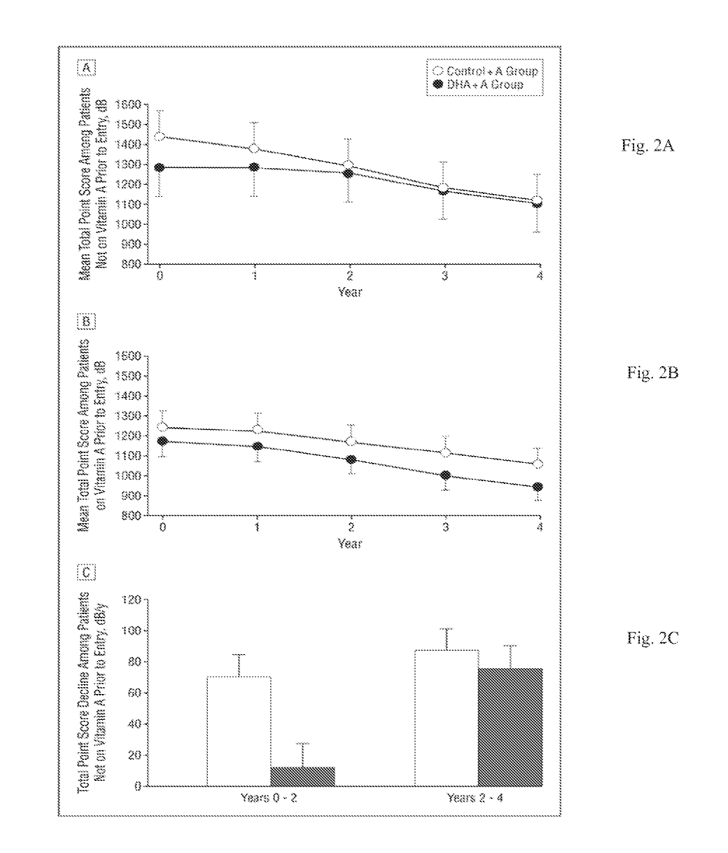

FIG. 2A shows values (mean±SE) of total visual field sensitivity (total point score for Humphrey Field Analyzer (HFA) 30-2 and 30/60-1 programs combined) by year among those not on vitamin A prior to entry in the DHA+A vs control+A groups. In these subgroups, the difference between the 2 curves was larger during years 1 and 2 and smaller during years 3 and 4. In contrast, as seen in FIG. 2B, among those on vitamin A prior to entry, the differences between the curves for the DHA+A vs control+A groups were not significantly different for either time period, although a slight divergence of the curves was noted particularly in years 3 and 4. FIG. 2C shows the annual rates (mean±SE) among those not on vitamin A prior to entry for years 0 to 2 and 2 to 4 for rate of total field sensitivity decline when comparing the DHA+A vs control+A groups. The rate of decline was significantly slower in the DHA+A vs control+A groups for years 0 to 2 (P=0.006), but was not significantly different for years 2 to 4 (P=0.57). Id.

Referring to Table 5 below, shows that among patients in the control+A group on vitamin A prior to entry, the rate of decline in visual field sensitivity over a 4-year interval for the central (30-2) condition was significantly related to the amount of dietary omega-3 fatty acid intake; those with an intake of at least 0.20 g/d had a 40% to 50% slower rate of decline compared with those with intake of less than 0.20 g/d (P=0.02). A similar result was seen for the total (HFA 30-2 and 30/60-1 combined) condition (P=0.05). Similar trends were seen for years 0 to 2 and 2 to 4, although the differences were only significant for the latter period (P=0.03). The same pattern was seen for the 30-2 and total conditions among those not on vitamin A prior to entry, but the differences were not significant (data not shown). Id.

TABLE 5
Annual Decline in Visual Field Sensitivity in the Control Group
as a Function of Dietary omega-3 Fatty Acid Intake Among
Patients on Vitamin A Prior to Entry*

| Dietary omega-3 | | | |
|---|---|---|---|
| Intake† | Years 0 to 4 | Years 0 to 2 | Years 2 to 4 |
| 30-2 Condition, | | | |
| dB | | | |
| <0.20 g/d | 39.2 ± 5.5 (35) | 32.9 ± 8.2 (35) | 44.5 ± 7.5 (35) |
| ≧0.20 g/d | 20.8 ± 5.7 (33) | 14.3 ± 8.4 (33) | 20.1 ± 7.7 (33) |
| P Value‡ | .02 | .12 | .03 |
| Total condition, | | | |
| dB§ | | | |
| <0.20 g/d | 57.8 ± 7.0 (35) | 44.7 ± 12.4 (35) | 72.9 ± 10.5 (35) |
| ≧0.20 g/d | 37.9 ± 7.2 (33) | 25.0 ± 12.7 (33) | 39.3 ± 10.8 (33) |
| P value‡ | .05 | .27 | .03 |

*Unless otherwise indicated, data are expressed as mean ± SE (number of patients sampled). Patients received control capsules plus 15 000 IU/d of vitamin A.
†Expressed as the mean of grams per day of all visits.
‡Based on PROC MIXED (SAS Institute Inc, Cary, NC) analysis.
§Total condition consists of the sum of 30-2 and 30/60-1 conditions when both are available.

The relevant teachings of all the references, patents and/or patent applications cited herein are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A nut oil composition for maintaining or improving health, said composition comprises two or more of the following:
a. a walnut oil in an amount ranging between about 10% and about 35% by weight;
b. an almond oil in an amount ranging between about 10% and about 35% by weight;
c. an avocado oil in an amount ranging between about 10% to about 35% by weight; and
d. a pistachio oil in an amount ranging between about 10% to about 35% by weight.

2. The nut oil composition of claim 1, further including a carrier or binder.

3. The nut oil composition of claim 1, further comprising a fatty acid selected from the group consisting of palmitic acid, palmitoleic acid, heptadecanoic acid, stearic acid, arachidic acid, eicosenoic acid, linoleic acid, linolenic acid, oleic acid and any combination thereof.

4. The nut oil composition of claim 4, wherein the carrier comprises a softgel capsule, a capsule, a caplet, a pill, liquid formulation or any combination thereof.

5. The nut oil composition of claim 1, wherein the ratio of walnut oil, the ratio of almond oil, the ratio of avocado oil and the ratio of pistachio oil in the composition is in the range of 1:1:1:1.

6. A nut oil composition, said composition comprises two or more of the following:
a. a walnut oil in an amount ranging between about 100 mg/mL and about 350 mg/mL;
b. an almond oil in an amount ranging between about 100 mg/mL and about 350 mg/mL;
c. an avocado oil in an amount ranging between about 100 mg/mL and about 350 mg/mL; and
d. a pistachio oil in an amount ranging between about 100 mg/mL and about 350 mg/mL.

7. A nut oil composition, said composition comprises:
a. a walnut oil in an amount ranging between about 10% and about 35% by weight;
b. an almond oil in an amount ranging between about 10% and about 35% by weight;
c. an avocado oil in an amount ranging between about 10% to about 35% by weight; and
d. a pistachio oil in an amount ranging between about 10% to about 35% by weight;
whereby the composition comprises fatty acids selected from the group consisting of palmitic acid, palmitoleic acid, heptadecanoic acid, stearic acid, arachidic acid, eicosenoic acid, linoleic acid, linolenic acid, oleic acid and any combination thereof.

8. The nut oil composition of claim 7, wherein the composition further comprises antioxidants, vitamins, minerals, phytonutrients, additional fatty acids or any combination thereof.

9. The nut oil composition of claim 7 further including vitamin E in an amount of between about 10% and about 15% by weight.

10. The nut oil composition of claim 7 further an amount of the omega-3 in the range of about 2.0% to about 10% of total lipids in composition; an amount of the omega-6 in the range of about 25% to about 45% of total lipids in composition; an amount of the omega-7 in the range of about 0.1% and about 5% of total lipids in composition; an amount of the omega-9 in the range about 40% and about 60% of total lipids in composition.

11. A nut oil composition, said composition comprises:
a. a palmitic acid in an amount ranging between about 5 to about 20% by weight;
b. a stearic acid in an amount ranging between about 0.5% to about 10% by weight;
c. an oleic acid in an amount ranging between about 40% and about 60% by weight;
d. a linoleic acid in an amount ranging between about 25% to about 45% by weight;
e. an alpha linolenic acid in an amount ranging between about 2% and about 10% by weight;
f. a palmitoleic acid in an amount ranging between about 0.1% and about 5% by weight;
g. a heptadecanoic acid in an amount ranging between about 0.1% and about 0.25% by weight;
h. an arachidic acid in an amount ranging between about 0.1% and about 0.25% by weight; and
i. an eicosenoic acid in an amount between about 0.1% and about 0.25% by weight.

12. A method of treating an individual with a high total cholesterol level (Total-C), a high blood lipid level or both in an individual, wherein the method comprises:
a. selecting an individual having a high total cholesterol level, a high blood lipid level or both;
b. administering to said individual an effective amount of nut oil composition comprising:
i. a walnut oil in an amount ranging between about 10% and about 35% by weight;
ii. an almond oil in an amount ranging between about 10% and about 35% by weight;
iii. an avocado oil in an amount ranging between about 10% to about 35% by weight; and
iv. a pistachio oil in an amount ranging between about 10% to about 35% by weight
wherein the nut oil composition decreases the total cholesterol level, blood lipid level or both as compared to the level prior to step b).

13. The method of claim 12, wherein the total cholesterol level, the blood lipid level or both is decreased by at least 5%.

14. The method of claim 12, wherein the nut oil composition is consumed daily, weekly, monthly or any combination thereof.

15. The method of claim 12, wherein the nut oil composition is consumed twice a day.

16. The method of claim 12, wherein the individual has a Total-C level of greater than about 200 mg/dL.

17. The method of claim 12, wherein the individual has a LDL-C level of greater than about 130 mg/dL.

18. A method of reducing retinal degeneration in an individual with retinitis pigmentosa, wherein the method comprises:
a. selecting an individual having retinitis pigmentosa;
b. administering to said individual an effective amount of nut oil composition comprising:
i. a walnut oil in an amount ranging between about 10% and about 35% by weight;
ii. an almond oil in an amount ranging between about 10% and about 35% by weight;
iii. an avocado oil in an amount ranging between about 10% to about 35% by weight; and
iv. a pistachio oil in an amount ranging between about 10% to about 35% by weight
wherein the nut oil composition reduces retinal degeneration as compared to the level prior to step b).

19. The method of claim 19, wherein retinal degeneration is reduced by at least 5%.

20. A method of reducing the decline in visual field sensitivity in an individual with retinitis pigmentosa, wherein the method comprises:
a. selecting an individual having retinitis pigmentosa;
b. administering to said individual an effective amount of nut oil composition comprising:
i. a walnut oil in an amount ranging between about 10% and about 35% by weight;
ii. an almond oil in an amount ranging between about 10% and about 35% by weight;
iii. an avocado oil in an amount ranging between about 10% to about 35% by weight; and
iv. a pistachio oil in an amount ranging between about 10% to about 35% by weight
wherein the nut oil composition reduces the decline of visual field sensitivity as compared to the level prior to step b).

21. The method of claim 20, wherein the decline in visual field sensitivity is reduced by at least 5%.
